(19) United States
(12) Patent Application Publication
(10) Pub. No.: US 2008/0188746 A1
(43) Pub. Date: Aug. 7, 2008

(54) Method and Apparatus for Automatic Gain Adjustment in Spectral Doppler

(75) Inventors: Thanasis Loupas, Psychiko (GR); Ashraf A. Saad, Redmond, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/817,842

(22) PCT Filed: Mar. 2, 2006
(86) PCT No.: PCT/IB06/50655

§ 371 Date: Sep. 5, 2007

Related U.S. Application Data
(60) Provisional application No. 60/659,608, filed on Mar. 8, 2005.

(51) Int. Cl. A61B 8/00

(52) U.S. Cl. 600/441

(57) ABSTRACT

A method for automatic gain adjustment in spectral (pulsed wave—PW, and/or continuous wave—CW) Doppler for medical ultrasound includes separating a two-dimensional (2D) array of spectral levels (spectrogram) to be analyzed into signal and noise subsets. For each of the signal and noise subsets, a delta gain is calculated for achieving a predetermined display-based design specification. Subsequently, the separate signal and noise delta gains are combined into a single delta gain value, which is then applied to subsequent spectral Doppler signals or the spectrogram data stored in image memory, depending on whether the spectral Doppler mode is in a live or frozen state.

The present embodiments relate generally to medical ultrasound systems and more particularly, to a method and apparatus for automatic gain adjustment in the spectral (pulsed wave—PW, or continuous wave—CW) Doppler modes of a medical ultrasound system.

Gain represents one of the most important and frequently manipulated spectral Doppler controls, since it has considerable impact on blood-flow/tissue-motion detectability, visualization and quantification. However, spectral Doppler gain requires frequent and careful adjustments throughout an exam, in order to maintain an optimum sonogram display in response to changes in the signal characteristics and noise properties. Changes in signal characteristics can occur, for example, as the sample volume is moved to a new vessel location. In addition, changes in noise properties can be affected, for example, by factors such as the sample volume size, PRF (Pulse Repetition Frequency) etc.

Accordingly, an improved method and ultrasound diagnostic imaging system for performing an optimization of the spectral Doppler gain control for overcoming the problems in the art is desired.

According to an embodiment of the present disclosure, automatic optimization of the spectral Doppler gain control addresses the time-consuming and tedious nature of manual gain adjustments. In one embodiment, the automatic optimization of the spectral Doppler gain control is implemented in the form of an AutoGain algorithm of an ultrasound diagnostic imaging system.

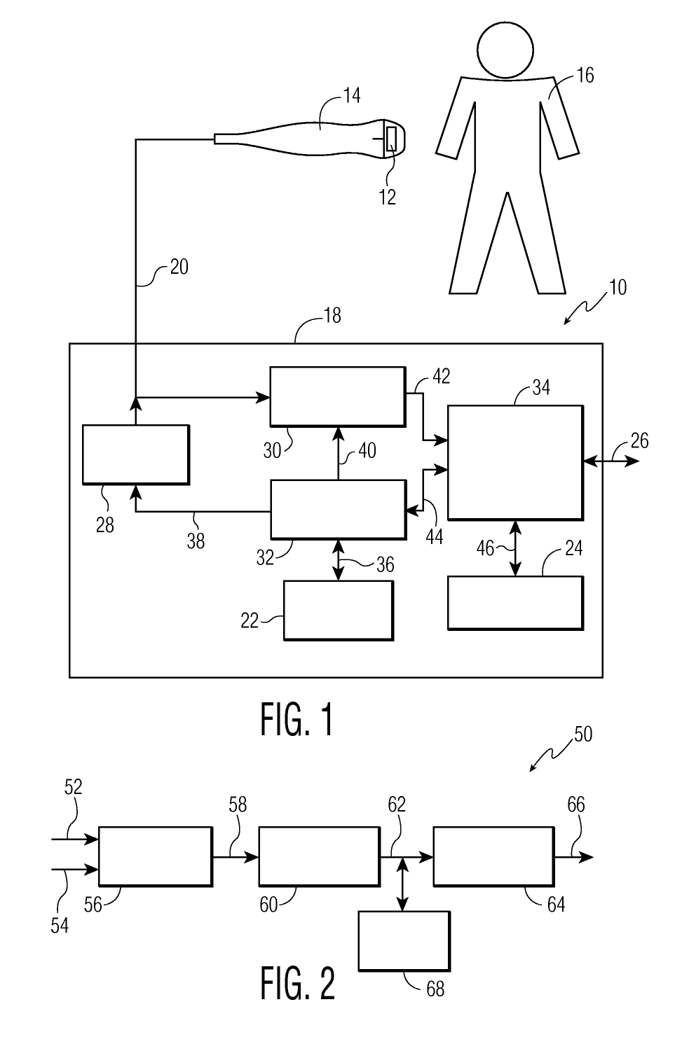

FIG. 1 is a partial block diagram view of an ultrasound diagnostic imaging system incorporating an AutoGain algorithm according to an embodiment of the present disclosure;

FIG. 2 is a simplified block diagram view of a spectral Doppler processing path of an ultrasound diagnostic imaging system in connection with an AutoGain algorithm according to an embodiment of the present disclosure;

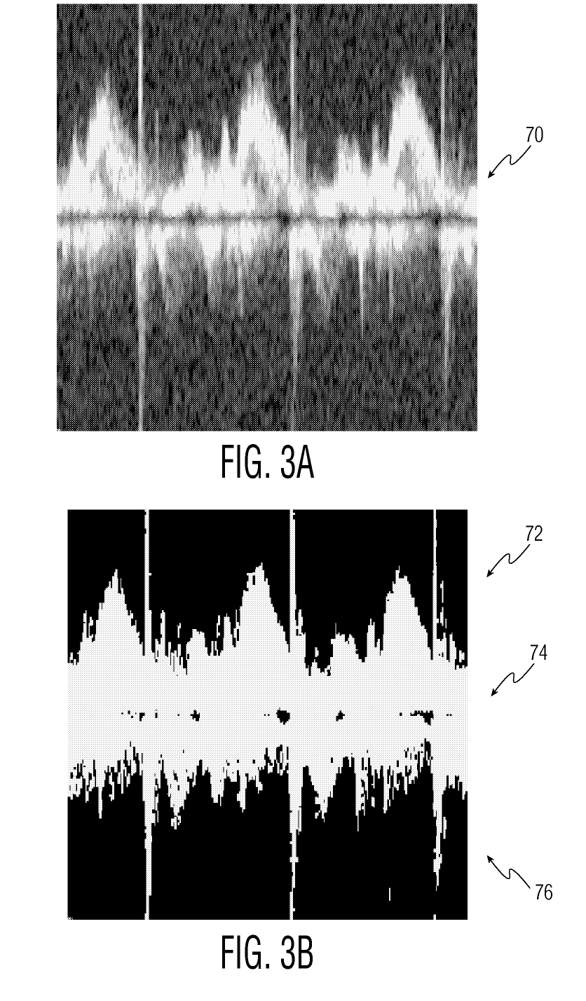

FIG. 3A is a grayscale display view of a Doppler spectrogram and FIG. 3B is a segmented version of the spectrogram of FIG. 3A, displayed as a binary image;

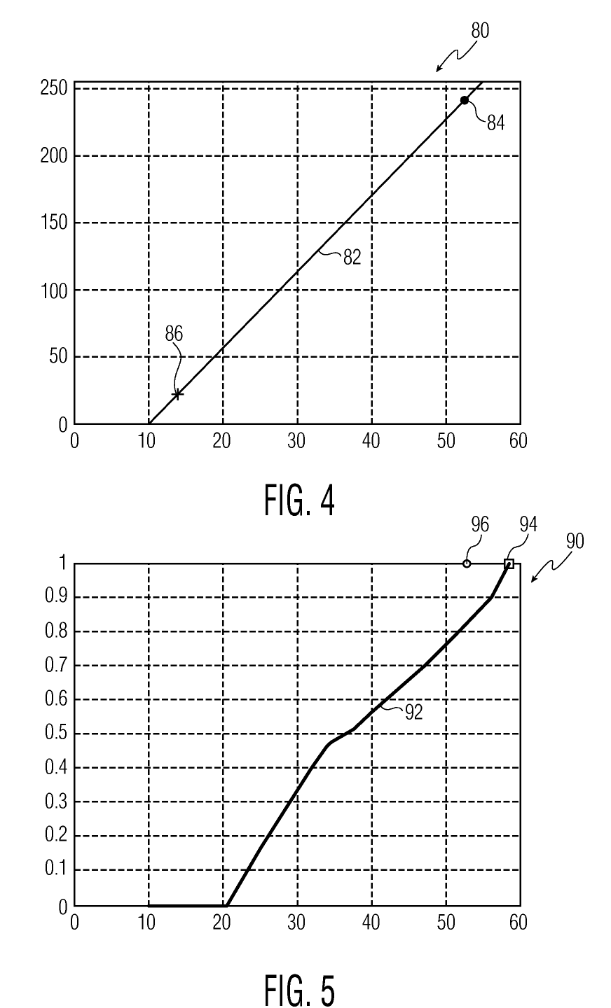

FIG. 4 is a graphical representation view of an overall Doppler map, which includes the effect of all stages in a Doppler processing path and specifies a correspondence between uncompressed spectral levels and grayscale levels used in a Doppler spectrogram display;

FIG. 5 is a graphical representation view of a Cumulative Distribution Function (CDF) corresponding to signal pixels versus a range of uncompressed spectral levels of the corresponding signal; and

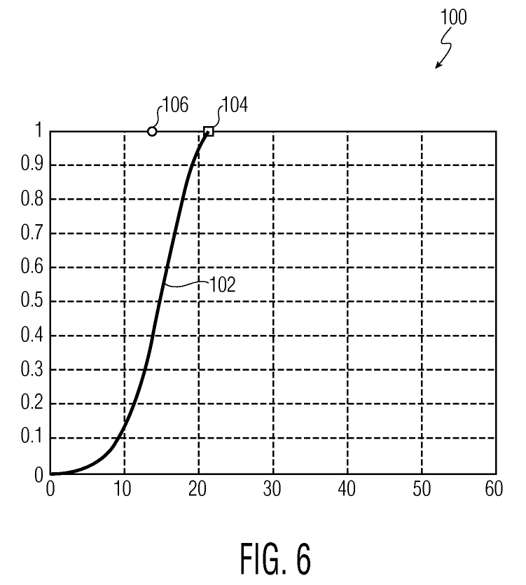

FIG. 6 is a graphical representation view of a Cumulative Distribution Function (CDF) corresponding to noise pixels versus a range of uncompressed spectral levels of the corresponding noise.

In the figures, like reference numerals refer to like elements. In addition, it is to be noted that the figures may not be drawn to scale.

FIG. 1 is a partial block diagram view of an ultrasound diagnostic imaging system 10 that incorporates an AutoGain algorithm according to an embodiment of the present disclosure. In connection with ultrasound diagnostic imaging system 10, an ultrasound transducer array 12 is disposed within a housing 14. The ultrasound transducer array 12 is adapted for being placed adjacent to or proximate an object of interest (or portion thereof) to be imaged, for example, a patient 16. The transducer array 12 can include, for example, any suitable transducer array, such as a 2D array, known in the art. In addition, the transducer can be configured for moving along a path, as may be desired, to scan the object to be imaged.

Ultrasound diagnostic imaging system 10 includes a control electronics unit 18. Ultrasound transducer array 12 couples to the control electronics unit 18 via a signal line 20. The control electronics unit 18 includes and/or interfaces with an input/output device 22 (such as a keyboard, mouse, touch screen, audio/voice input, toggle switch, push-button switch, or the like) and a display device 24, the control electronics unit providing imaging data signals to the video display for visual display. The control electronics unit 18 may further provide ultrasound image data to other devices (not shown), such as a printer, a mass storage device, computer network (i.e., for remote data storage, analysis, and/or display), etc., via data signal transmissions on signal line 26 suitable for use by the destination device. In one embodiment, the control electronics unit 18 further includes a transmitter 28 (e.g. a transmit beamformer), digital beamformer 30 (e.g., a receive beamformer), a system controller 32, and an image processor 34.

The system controller 32 couples to the I/O device 22 via signal line 26. The system controller 32 also provides appropriate transmit beamformer control signals to transmitter 28 via signal line 38. The transmit beamformer control signals are configured for providing the desired beam steering by the ultrasound transducer array as discussed further herein. Responsive to the transmit beamformer control signals, transmitter 28 provides corresponding ultrasound transducer control signals to ultrasound transducer array 12 via signal line 20.

In addition, the system controller 32 also provides appropriate receive beamformer control signals to digital beamformer 30 via signal line 40. The receive beamformer control signals are configured for providing a desired beamforming according to the embodiments of the present disclosure, as discussed further herein. Digital beamformer 30 provides ultrasound image data to image processor 34 via signal line 42. Furthermore, system controller 32 couples to image processor/memory 34 via signal line 44. Responsive to control signals from system controller 32 and responsive to ultrasound image data from digital beamformer 30, image processor/memory 34 provides image data to display device 24 via signal line 46, the image data being suitable for use by display device 24. The components of electronic unit 18 can include any suitable components known in the art for carrying out various functions as discussed herein.

The transmission of ultrasound beams is controlled by transmitter 28. Transmitter 28 controls the phasing and time of actuation of each of the elements of the array transducer 12 so as to transmit each beam from a predetermined origin along the array and at a predetermined angle or steering direction, and focus. The echoes returned from along each scanline are received by the elements of the array, digitized as by analog to digital conversion (not shown), and coupled to digital beamformer 30. The digital beamformer 30 delays and sums the echoes from the array elements to form a sequence of focused, coherent digital echo samples along each scanline. The transmitter 28 and beamformer 30 are operated under control of system controller 32, which in turn is responsive to the settings of controls of a user interface 22 operated, for example, by a user of the ultrasound system or according to an automated protocol. The system controller 32 controls the transmitter 28 to transmit the desired number of scanline groups at the desired angles, focuses, transmit energies and frequencies. The system controller 32 also controls the digital beamformer 30 to properly delay and combine the received echo signals for the apertures and image depths used.

In accordance with the embodiments of the present disclosure, the image data is presented in a display format by image processor 34, wherein the image processor/memory can include an image rendering processor suitable for the requirements of a particular ultrasound diagnostic imaging application. Image data is rendered into a display presentation. The rendering can be controlled by rendering control signals selected by the user interface 22 and applied to the processor 34 by the system controller 32.

FIG. 2 is a simplified block diagram view of a spectral Doppler processing path of an ultrasound diagnostic imaging system 10 in connection with an AutoGain algorithm according to one embodiment of the present disclosure. In this embodiment, the AutoGain algorithm assumes a spectral Doppler processing path 50. The following description of the spectral Doppler processing path 50 of FIG. 2 highlights various assumptions made by the AutoGain algorithm.

The Doppler signals include an I component 52 and a Q component 54, which together form I and Q pairs. The I and Q pairs of the Doppler signal undergo spectral analysis via spectral analysis stage 56. Spectral analysis stage 56 produces spectral power estimates on output 58, subsequently referred to herein as “uncompressed spectral levels”. Note that for Doppler signals corresponding to blood flow, the Doppler signals would go through a wall filtering stage prior to spectral analysis. In contrast, wall filtering is typically not used when the goal is to display and quantify the velocities associated with moving tissue, for example, in a Tissue Doppler mode.

The uncompressed spectral levels on output 58 are input to compression stage 60. Compression stage 60 compresses the spectral levels to accommodate the limited dynamic range of subsequent stages. The compression stage 60 provides compressed spectral levels on output 62.

The compressed spectral levels on output 62 are fed as inputs to one of an available graylevel or chroma map stage, for example, gray map stage 64, to produce RGB triplets on output 66 for driving the Spectral Doppler display.

In addition to being fed to the spectral Doppler display, the compressed spectral levels are also stored in image memory 68 as a 2D array. The rows and columns of the 2D array of compressed spectral levels correspond to Doppler frequency and time, respectively. Note, however, that while the image memory has been illustrated for receiving output from compression stage 60, image memory 68 could be moved to a different part of the block diagram of FIG. 2. For example, image memory 68 could appear between the spectral analysis and compression blocks, 56 and 60, respectively, without affecting the essence of AutoGain algorithm embodiments of the present disclosure.

According to one embodiment of the present disclosure, in response to an invocation or activation event, the AutoGain algorithm accesses a prescribed amount of spectral Doppler data stored in image memory, analyzes those data with regards to separate noise and signal gain optimization criteria, and derives an “optimum” delta gain factor to be applied on subsequent spectral Doppler signals in order to meet the gain optimization criteria. The AutoGain algorithm derives the “optimum” delta gain factor as a function of the analyzed data. In addition, the prescribed amount of spectral Doppler data could represent, for example, the last one to two (1-2) seconds of spectral Doppler data prior to invocation of the AutoGain algorithm. Furthermore, the prescribed amount of spectral Doppler data could also represent any other amount of data deemed necessary for the particular spectral Doppler AutoGain implementation.

The invocation or activation event corresponds to any suitable action or event according to a given discrete/continuous activation model that may be used in connection with the AutoGain algorithm or method of the present disclosure. The AutoGain algorithm can be activated or invoked in a number of different ways. More specifically, AutoGain activation can occur on a discrete basis as a result of an explicit user action, for example, by the pushing of a dedicated key, a voice command, etc. on the ultrasound diagnostic imaging system. In addition, AutoGain activation can occur on a continuous basis by continuously running the AutoGain algorithm as a background process and accepting new gain estimates in response to a new gain estimate being sufficiently different than a currently used gain (i.e., with respect to a threshold value). Furthermore, additional logic may be used to detect conditions such as saturation which may require multiple iterations of the Doppler AutoGain algorithm to converge to a truly optimum gain estimate. Moreover, AutoGain algorithm activation can occur as a result of a specific event, such as, a transition from an imaging to a spectral Doppler mode on an ultrasound diagnostic imaging system.

In one embodiment, the AutoGain algorithm includes: i) segmenting a Doppler spectrogram to be analyzed into signal and noise subsets, ii) for each of the signal and noise subsets of the Doppler spectrogram, calculating a delta gain needed to achieve prescribed display-based design specifications, and iii) applying prescribed rules to combine the separate signal and noise delta gains into a overall delta gain value to be applied to the entire (signal plus noise) spectral Doppler data.

In connection with the preceding paragraph, the prescribed display-based design specifications for the signal or noise subsets are expressed in terms of pairs of values. For example, the pairs of values for the signal subsets can be represented by {DesSigPrc, DesSigMapLev}. Likewise, the pairs of values for the noise subsets can be represented by {DesNoisPrc, DesNoisMapLev}. The pairs of values for the prescribed display-based design specifications specify that the data of interest (i.e., signal or noise) should be such that their design percentile (DesPrc) is mapped to a design map level (DesMapLev) on the spectral Doppler display.

The design specifications for noise are based on an assumption that the upper one to ten percent (1-10%) of the noise pixels should be just above display visibility. This corresponds, for example, to graylevels of around ten to twenty (10-20) out of 256 graylevels. In contrast, the design specifications for signal are based on an assumption that the upper one to ten percent (1-10%) of the signal pixels should be just above display saturation. This corresponds, for example, to graylevels of around two hundred to two hundred and forty (200-240) out of 256 graylevels.

In order to calculate the signal delta gain, the AutoGain algorithm first finds the uncompressed spectral level (DesSigUncompSpectrLev) of signal that corresponds to the design map level (DesSigMapLev). The AutoGain algorithm achieves this by calculating the inverse of the transformations defined by the Compression and Gray Map stages 60 and 64, respectively, of FIG. 2. Preferably, DesSigUncompSpectrLev is expressed in decibel units, that is, DesSigUncompSpectrLev=10*LOG10(DesSigUncompSpectrLev_Linear) dB. The AutoGain algorithm then finds the uncompressed spectral level (CurSigUncompSpectrLev) that currently corresponds to the DesSigPrc percentile of the signal pixels. Preferably, CurSigUncompSpectrLev is also expressed in decibel units. The AutoGain algorithm finds the CurSigUncompSpectrLev by means of a Cumulative Distribution Function (CDF) of the signal pixels as a function of the uncompressed spectral levels. This is obtained either by forming the signal CDF as a function of the range of values stored in image memory and then using inverse transformations to translate the range of values stored in image memory into uncompressed spectral levels, or by inverse transformation of the signal values stored in image memory into the corresponding uncompressed spectral levels followed by the formation of the signal CDF as a function of the uncompressed spectral levels. Finally, the AutoGain algorithm finds the signal delta gain as: DeltaGainSig=DesSigUncompSpectrLev−CurSigUncompSpectrLev dB. In order to calculate the noise delta gain, the AutoGain algorithm similarly follows the signal approach outlined above. That is, the AutoGain algorithm first finds the uncompressed spectral level of noise (DesNoisUncompSpectrLev) in dB units that corresponds to the design map level (DesNoisMapLev). The AutoGain algorithm then finds the uncompressed spectral level (CurNoisUncompSpectrLev) in dB units that currently corresponds to the DesNoisPrc percentile of the noise pixels. Finally, the AutoGain algorithm derives the noise delta gain from the expression:

DeltaGainNois=DesNoisUncompSpectrLev−CurNoisUncompSpectrLev dB.

As discussed herein, an optimum gain estimation for either the signal or the noise subsets involves forming the histogram of those sonogram pixels belonging to the subset of interest, calculating the histogram's x-th percentile by means of the cumulative distribution function, and then computing the delta gain (or multiplication factor) which, when applied to the Doppler data prior to the spectral analysis stage, forces the x-th percentile to be mapped to the N-th gray level on the display. The optimization criteria can be kept relatively simple (i.e., the 95-th signal percentile mapped to gray level of, for example, 230 to make sure that the majority of the signal pixels are below saturation, and the 95-th noise mapped to gray level of, for example, 10 to make sure that noise pixels are just visible). More elaborate criteria, possibly involving multiple percentile-to-gray level specifications, can be developed in response to specific clinical requirements during integration of the AutoGain algorithm within the particulars of a given ultrasound application. In addition, the calculations discussed herein can be configured to take explicitly into account the effect of any other modules (i.e. filtering, decimation, . . . ) not shown in the simplified block diagram of FIG. 2.

The above procedures of the AutoGain algorithm according to the embodiments of the present disclosure are further explained with use of the remaining figures and examples presented below.

FIG. 3A is a grayscale display view of a Doppler spectrogram 70, used as input to the AutoGain algorithm. The horizontal axis of the spectrogram corresponds to a temporal duration of two seconds. FIG. 3B is a segmented version 72 of the spectrogram of FIG. 3A, displayed as a binary image (foreground: signal; background: noise). In other words, in FIG. 3A, pixels classified as signal are shown in white (foreground) 74 and pixels classified as noise are shown in black (background) 76.

FIG. 4 is a graphical representation view of the overall Doppler map 80, which takes into account the entire Doppler signal path including the Compression and Gray Map stages to define the correspondence between uncompressed spectral levels and grayscale levels on the spectral Doppler display, as indicated by the line 82. Note that, due to their large dynamic range, the uncompressed spectral levels are plotted in a decibels scale 10*Log10 (Uncompressed Spectral Level). Also, note that a correspondence between uncompressed spectral levels and grayscale levels can be established even when a chroma map is used instead of a gray map, i.e. a color map specifying triplets of red (R), green (G) and blue (B) values. In this case, a graylevel-equivalent value G is obtained by means of a combination of the R, G, and B components. To provide specific examples, which will be used later to explain the AutoGain algorithm, two markers are also shown on FIG. 4. The first marker 84 is an asterisk, and indicates that the uncompressed spectral level of 52.4 dB will be mapped to grayscale level 240. The second marker 86 is a cross, and indicates that the uncompressed spectral level of 13.5 dB will be mapped to grayscale level 20.

FIG. 5 is a graphical representation view 90 of a cumulative distribution function of the signal pixels from the two-second (2-sec) Doppler spectrogram of FIG. 3. The signal CDF, or cumulative histogram, illustrated by line 92, is obtained by calculating the histogram of the signal pixels as a function of the uncompressed spectral levels (expressed in decibel scale), and then integrating this histogram starting from zero. Two markers are overlayed on the plot of FIG. 5. The first marker 94 is a square, and indicates that the 99^{th} percentile of the signal pixels currently corresponds to an uncompressed spectral level of 58 dB. The second marker 96 is a circle and defines a point with an x-coordinate of 52.4 dB and a y-coordinate of 0.99 (or 99%, i.e. 99^{th} percentile).

FIG. 6 is a graphical representation view 100 of a cumulative distribution function (CDF) of the noise pixels from the two-second (2-sec) Doppler spectrogram of FIG. 3. The noise CDF, or cumulative histogram, illustrated by line 102, is obtained by calculating the histogram of the noise pixels as a function of the uncompressed spectral levels (expressed in decibel scale), and then integrating this histogram starting from zero. Two markers are overlayed on the plot of FIG. 6. The first marker 104 is a square, and indicates that the 99^{th} percentile of the noise pixels currently corresponds to an uncompressed spectral level of 21 dB. The second marker 106 is a circle and defines a point with an x-coordinate of 13.5 dB and a y-coordinate of 0.99 (or 99%, i.e. 99^{th} percentile).

As an example of estimating the signal delta gain, let's assume that the signal design specifications are DesSigPrc=99 and DesSigMapLev=240, and that the overall Doppler map used is the one plotted in FIG. 4. From this figure, it can be deduced that the gray level DesSigMapLev=240 corresponds to an uncompressed spectral level DesSigUncompSpectrLev=52.4 dB. On the other hand, from the signal CDF plotted in FIG. 5 it can be deduced that the DesSigPrc=99^{th} signal percentile currently corresponds to an uncompressed spectral level CurSigUncompSpectrLev=58 dB. Therefore, a signal delta gain of −5.6 dB is needed to meet the signal design specification. In other words, DeltaGainSig=DesSigUncompSpectrLev−CurSigUncompSpectrLev=52.4−58 dB=−5.6 dB. Accordingly, the signal delta gain of −5.6 dB enables the signal design specifications of mapping the 99^{th} percentile of signal pixels to a grayscale level of 240 on the Doppler spectrogram display to be met.

As an example of estimating the noise delta gain, let's assume that the noise design specifications are DesNoisPrc=99 and DesNoisMapLev=20, and that the Doppler map used is the one plotted in FIG. 4. From this figure, it can be deduced that the gray level DesNoisMapLev=20 corresponds to an uncompressed spectral level DesNoisUncompSpectrLev=13.5 dB. On the other hand, from the noise CDF plotted in FIG. 6 it can be deduced that the DesNoisPrc=99^{th} noise percentile currently corresponds to an uncompressed spectral level CurNoisUncompSpectrLev=21 dB. Therefore, a noise delta gain of −7.5 dB is needed to meet the noise design specification. In other words, DeltaGainNois=DesNoisUncompSpectrLev−CurNoisUncompSpectrLev=13.5−21 dB=−7.5 dB. Accordingly, the noise delta gain of −7.5 dB enables the noise design specifications of mapping the 99^{th} percentile of noise pixels to a grayscale level of 20 on the Doppler spectrogram display to be met.

In alternate embodiments, the signal and/or noise design specifications can, in general, include more than one pairs of percentile vs. Doppler map level values. For example, the signal design specifications can be expressed in terms of N pairs of values, {DesSigPrcₙ, DesSigMapLevₙ} where n=1, 2, . . . , N, specifying optimality criteria for different segments of the signal range (low-level, mid-level, high-level, . . . ), with the resulting signal delta gains DeltaGainSigₙ (n=1, 2, . . . , N) combined to produce a single signal delta gain by means of simple rules (DeltaGainSig=MAX{DeltaGainSigₙ}, MIN{DeltaGainSigₙ}, etc), weighted sums

$$( DeltaGainSig = {\sum }_{n = 1}^{N} {C}_{n} {DeltaGainSig}_{n} )$$

or other appropriate approaches.

Further with the AutoGain algorithm of the present disclosure, the signal and noise delta gains can be combined into an overall delta gain. Combining the signal and noise delta gains into the overall delta gain can be accomplished by means of one or more different algorithms, wherein a selection of the particular algorithm depends on factors such as the type of data to be analyzed (for example, peripheral vascular, cardiac blood flow, or cardiac Tissue Doppler) and user-specific preferences. Two approaches, that have been found useful in specific applications, are outlined below.

In a first approach, the overall delta gain is determined as a weighted combination of the individual delta gains, according to the expression;

DeltaGain=a*DeltaGainSig+b*DeltaGainNois,

where a and b are application-specific and possibly data-dependent coefficients. For example, coefficients a and b can be determined by combining simple rules such as:

IF (DeltaGainSig< DeltaGainNois)
{
a=1;
b=0;
}
ELSE {
a=0.25;
b=0.75;
}

In addition, for some cases, coefficients a and b can be determined in terms of data-dependent features such as Signal-to-Noise Ratio (SNR) and application-specific look-up tables (LUTs), such as:

a=LUTₐ(SNR);

b=LUT_{b}(SNR).

In a second approach, the overall delta gain is determined by the noise delta gain, and the characteristics of the Compression or Gray Map stages are then modified accordingly to match the signal design specifications. To give an example, and using the specific values from FIGS. 4 thru 6 mentioned above,

DeltaGain=DeltaGainNois=−7.5 dB.

After DeltaGain is applied, the DesSigPrc=99^{th} percentile corresponds to a new uncompressed spectral level:

$$\begin{matrix}NewSigUncompSpectrLev = CurSigUncompSpectrLev + \\ DeltaGain \\ = 58 - 7.5 dB \\ = 50.5 dB .\end{matrix}$$

Accordingly, the Compression and/or Gray Map stages are modified to make sure that the overall Doppler Map of FIG. 3 passes through the point defined by the x-coordinate NewSigUncompSpectrLev=50.5 dB and the y-coordinate DesSigmapLev=240.

Accordingly, an AutoGain algorithm has been disclosed herein that analyzes spectral Doppler sonogram values stored in an image memory. The AutoGain algorithm derives an appropriate gain value corresponding to an optimum representation of the sonogram on the system's spectral Doppler display. The analysis considers the signal as well as noise components of the sonogram. Furthermore, the analysis takes into account a currently selected gray or chroma map and current spectral compression characteristics in order to achieve a close approximation of a manual gain optimization performed, for example, by expert users. Moreover, the algorithm can be applied on live or frozen Spectral Doppler data.

With live spectral Doppler, the AutoGain algorithm analyzes the last x seconds of sonogram data stored in image memory, and performs the following operations: a) segments sonogram data into signal and noise subsets, based on knowledge of the noise statistics (theoretically-derived, stored in lookup tables, or dynamically estimated by means of histogram analysis and image processing techniques); b) estimates signal gain so that the signal subset matches the specified display-based signal optimization criteria (for example, to map a specific percentile of the signal pixels to a gray level just below saturation); c) estimates noise gain so that the noise subset matches the specified display-based noise optimization criteria (for example, to map a specific percentile of the noise pixels to a gray level just above visibility); and d) combines the signal and noise gains into an overall gain value, by utilizing specific rules and/or data-driven lookup tables.

Alternatively, the overall gain is determined by the noise gain, and then the signal design specifications are met by appropriate modification of the compression and/or gray map stages. The automatically estimated optimum gain is then communicated, for example, via the electronic control unit, to the ultrasound diagnostic imaging system, which updates the front-end and/or back-end Doppler gain values, accordingly. The above cycle (data analysis, gain estimation, gain application) may be repeated for a number of predefined times, to allow gradual convergence towards the optimum value in challenging cases such as heavy saturation.

In addition to live spectral Doppler, the AutoGain algorithm can also be used while spectral Doppler is frozen. The main difference is that the optimum gain is now applied to the spectrogram data already stored in image memory. In the case of frozen-state operation, the sonogram data analyzed by the AutoGain algorithm can have an arbitrary duration/position relative to the data stored in image memory (i.e. all the data stored in image memory, only the displayed portion of the spectral data, or any arbitrary portion of the image memory spectral data). Furthermore, multiple disjoint segments of sonogram data can be analyzed by the AutoGain algorithm, in which case the resulting multiple optimum gains can be combined into a single optimum value to be applied to the whole data stored in image memory, or each of the multiple gains can be individually applied to those image memory data segments used as inputs for the specific gain's derivation.

According to another embodiment, ultrasound diagnostic imaging system 10 further includes computer software configured, using programming techniques known in the art, for carrying out the various functions and functionalities of the AutoGain algorithm as described herein. In particular, responsive to instructions stored on a computer readable media and executable by a processor, the processor operates to carry out the AutoGain algorithm.

The embodiments of the present disclosure also include computer software or a computer program product. The computer program product includes a computer readable media having a set of instructions for carrying out the method of the AutoGain algorithm as described and discussed herein. The computer readable media can include any suitable computer readable media for a given ultrasound diagnostic imaging system application. Still further, the computer readable media may include a network communication media. Examples of network communication media include, for example, an intranet, the Internet, or an extranet.

Although only a few exemplary embodiments have been described in detail above, those skilled in the art will readily appreciate that many modifications are possible in the exemplary embodiments without materially departing from the novel teachings and advantages of the embodiments of the present disclosure. For example, the embodiments of the present disclosure can be applied to any ultrasound scanner supporting spectral Doppler. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

In addition, any reference signs placed in parentheses in one or more claims shall not be construed as limiting the claims. The word “comprising” and “comprises,” and the like, does not exclude the presence of elements or steps other than those listed in any claim or the specification as a whole. The singular reference of an element does not exclude the plural references of such elements and vice-versa. One or more of the embodiments may be implemented by means of hardware comprising several distinct elements, and/or by means of a suitably programmed computer. In a device claim enumerating several means, several of these means may be embodied by one and the same item of hardware. The mere fact that certain measures are recited in mutually different dependent claims does not indicate that a combination of these measures cannot be used to an advantage.

1. A method for automatic gain adjustment (AutoGain) in spectral Doppler for an ultrasound imaging system, the AutoGain method comprising:
separating a Doppler spectrogram of spectral Doppler data into signal and noise array subsets, the Doppler spectrogram including a two-dimensional (2D) array of spectral levels to be analyzed;
for each of the signal and noise array subsets, determining a delta gain for achieving predetermined display-based design specifications; and
combining the separate signal and noise delta gains into an overall delta gain, the overall delta gain for being applied to spectral Doppler data prior to display of the spectral Doppler data on a display.

2. The method of claim 1, wherein the predetermined display-based design specifications for the signal or noise subsets are expressed in terms of pairs of values, the pairs of values specifying that for given signal or noise subset data of interest, a signal or noise design percentile (DesPrc) is mapped to a corresponding signal or noise design map level (DesMapLev) on the spectral Doppler display.

3. The method of claim 1, wherein the design specifications for the signal array subset are based on an assumption that an upper one to ten percent of signal pixels should be just above display saturation.

4. The method of claim 2, wherein determining a signal delta gain (DeltaGainSig) includes finding the uncompressed spectral level of signal in dB units (DesSigUncompSpectrLev) that corresponds to the signal design map level (SigDesMapLev), finding the uncompressed signal spectral level in dB units (CurSigUncompSpectrLev) that currently corresponds to the signal design percentile (DesSigPrc) of the signal pixels, and calculating the difference between the DesSigUncompSpectrLev and CurSigUncompSpectrLev.

5. The method of claim 2, further wherein the design specifications for the signal subset include N pairs of values, (DesSigPrcₙ, DesSigMapLevₙ) where n=1, 2, . . . , N, specifying optimality criteria for different segments of a signal range, corresponding to one or more of low-level, mid-level, or high-level, with the resulting signal delta gains DeltaGainSigₙ (n=1, 2, . . . , N) combined to produce a single signal delta gain.

6. The method of claim 1, wherein the design specifications for the noise array subset are based on an assumption that an upper one to ten percent of noise pixels should be above display visibility.

7. The method of claim 2, wherein determining a noise delta gain (DeltaGainNois) includes finding the uncompressed spectral level of noise in dB units (DesNoisUncompSpectrLev) that corresponds to the noise design map level (NoisDesMapLev), finding the uncompressed noise spectral level in dB units (CurNoisUncompSpectrLev) that currently corresponds to the noise design percentile (DesNoisPrc) of the noise pixels, and calculating the difference between the DesNoisUncompSpectrLev and CurNoisUncompSpectrLev.

8. The method of claim 2, further wherein the design specifications for the noise subset include N pairs of values, (DesNoisPrcₙ, DesNoisMapLevₙ) where n=1, 2, . . . , N, specifying optimality criteria for different segments of a noise range, corresponding to one or more of low-level, mid-level, or high-level, with the resulting noise delta gains DeltaGainNoisₙ (n=1, 2, . . . , N) combined to produce a single noise delta gain.

9. The method of claim 1, wherein combining includes applying prescribed rules to combine the separate signal and noise delta gains into the overall delta gain value.

10. The method of claim 9, wherein the prescribed rules are a function of a type of ultrasound data to be analyzed or user-specific preferences.

11. The method of claim 9, wherein the prescribed rules include determining the overall delta gain value as a weighted combination of the individual signal and noise delta gains.

12. The method of claim 11, wherein the weighted combination further includes coefficients determined as a function of data-dependent features, the data-dependent features including signal-to-noise ratios (SNRs) and application specific look-up tables (LUTs).

13. The method of claim 9, wherein the prescribed rules include determining the overall delta gain as a function of the noise delta gain, and modifying characteristics of a compression or mapping accordingly to match the signal design specifications.

14. The method of claim 1, further comprising:
applying the overall delta gain value to spectral Doppler data for driving the spectral Doppler display.

15. The method of claim 1, further comprising:
activating the AutoGain method in response to an activation event, wherein the activation event includes one of a discrete event, a continuous event, or a combination of discrete and continuous events.

16. The method of claim 15, wherein the discrete event includes an explicit user action or a transition from an imaging mode to a spectral Doppler mode of an ultrasound system.

17. The method of claim 15, wherein the continuous event includes the AutoGain method continuously running as a background process and accepting new overall delta gain estimates in response to a new overall delta gain being sufficiently different than a currently used overall delta gain, with respect to a threshold value.

18. The method of claim 15, wherein the continuous event includes multiple iterations of the AutoGain method to converge to a new overall delta gain that is more optimal than a previous overall delta gain.

19. The method of claim 1, wherein the Doppler spectrogram includes on the order of a few seconds of live spectral Doppler data.

20. The method of claim 1, wherein the Doppler spectrogram includes spectral Doppler data previously stored in an image memory, the AutoGain method further comprising:
analyzing the previously stored spectral Doppler data, corresponding to frozen-state operation, further wherein the previously stored spectral Doppler data can have one of an arbitrary duration or position relative to overall spectral Doppler data stored in the image memory.

21. The method of claim 20, further comprising:
analyzing multiple segments of previously stored spectral Doppler data, wherein the multiple segments can include two or more fractions of spectral Doppler data stored in the image memory, shifted relative to each other to cover the entire image memory, further wherein a) resulting multiple optimum delta gains can be combined into a single optimum delta gain value to be applied to all the spectral Doppler data stored in the image memory, or b) each of the multiple optimum delta gains can be individually applied to corresponding ones of the image memory data segments used as inputs for deriving a corresponding delta gain.

22. An ultrasound imaging system including automatic gain adjustment (AutoGain) in spectral Doppler, said system comprising:
an ultrasound transducer array; and
an electronic control unit coupled to the ultrasound transducer array for generating a Doppler spectrogram of spectral Doppler data, said electronic control unit configured for (a) separating the Doppler spectrogram into signal and noise array subsets, the Doppler spectrogram including a two-dimensional (2D) array of spectral levels to be analyzed, (b) for each of the signal and noise array subsets, determining a delta gain for achieving predetermined display-based design specifications, and (c) combining the separate signal and noise delta gains into an overall delta gain, the overall delta gain for being applied to spectral Doppler data prior to display of the spectral Doppler data on a display.

23. A computer program product comprising:
computer readable media having a set of instructions for carrying out automatic gain adjustment (AutoGain) in spectral Doppler, the instructions being executable by a processor for:
(a) separating a Doppler spectrogram of spectral Doppler data into signal and noise array subsets, the Doppler spectrogram including a two-dimensional (2D) array of spectral levels to be analyzed,
(b) for each of the signal and noise array subsets, determining a delta gain for achieving predetermined display-based design specifications, and
(c) combining the separate signal and noise delta gains into an overall delta gain, the overall delta gain for being applied to spectral Doppler data prior to display of the spectral Doppler data on a display.
